(19) United States
(12) Patent Application Publication
(10) Pub. No.: US 2021/0136531 A1
(43) Pub. Date: May 6, 2021

(54) SYSTEM AND METHOD FOR AN EMERGENCY COMMUNICATION AND REMOTELY ACTIVATED EMERGENCY ASSIST DEVICE

(71) Applicants: Paul Dorian, Toronto (CA); Paul Angaran, Unionville (CA); Steven C. Brooks, Kingston (CA)

(72) Inventors: Paul Dorian, Toronto (CA); Paul Angaran, Unionville (CA); Steven C. Brooks, Kingston (CA)

(21) Appl. No.: 17/090,432

(22) Filed: Nov. 5, 2020

Related U.S. Application Data
(60) Provisional application No. 62/931,146, filed on Nov. 5, 2019.

(51) Int. Cl. H04W 4/029; H04B 7/06; A61N 1/39; G01S 19/47; G09B 19/00

(52) U.S. Cl. CPC H04W 4/029; H04B 7/06; G09B 19/003; G01S 19/47; A61N 1/39044

(57) ABSTRACT

Various embodiments are provided for an emergency assistance device for facilitating the delivery of emergency equipment to an emergency site, wherein the device is physically associated with the emergency equipment and the device comprises a communication unit for sending and receiving electronic messages; at least one alert device for providing an alert to at least one passerby in visual or audible range of the at least one alert device; a GPS sensor; at least one motion sensor; memory comprising mapping software; and a processor that is operatively coupled to the communication unit, the at least one alert device, the memory, the at least one motion sensor and the GPS sensor, wherein the processor is configured to: control the at least one alert device to generate an alert when the communication unit receives a remote activation electronic message; generate and provide direction instructions to the at least one passerby using the GPS sensor and the mapping software when the processor detects, via the at least one motion sensor, that the emergency assistance device is picked up or being moved by the at least one passerby; and provide usage instructions for using the emergency equipment at the emergency site when the processor determines that the device has arrived at the emergency site.

### CROSS-REFERENCE TO RELATED APPLICATION

This application claims the benefit of U.S. Provisional Patent Application No. 62/931,146 filed Nov. 5, 2019; the entire contents of Patent Application 62/931,146 is hereby incorporated by reference.

### FIELD

At least one embodiment is described herein that generally relates to a method and system to facilitate the most rapid possible delivery of emergency or safety equipment to a victim requiring assistance.

### BACKGROUND

Sudden cardiac death is common and survival rates are poor. In fact, every 15 minutes, someone in Canada suffers an out-of-hospital cardiac arrest and there are about 400,000 cardiac arrest annually in North America. The mortality from unexpected sudden out-of-hospital cardiac arrest is high, ranging from 80-95% in various settings. Most of these cardiac arrests happen because there is a heart rhythm disturbance known as ventricular fibrillation, where the only effective treatment is defibrillation. The victim usually dies unless they receive a life-saving electrical shock from a defibrillator within 5-10 minutes, such as from an Automated External Defibrillator (AED). Up to 25% of cardiac arrests occur in public locations, or in some private locations that are within easy access of public access defibrillators (AEDs) located in public buildings, multi-user facilities such as sports clubs, restaurants, stores, and other public venues, etc.

The best predictors of outcome following cardiac arrest is the time interval from when the collapse first occurred (i.e. the first occurrence of cardiac arrest) to when a defibrillator is first connected to the victim, as well as the time interval to the delivery of effective CPR. These two time intervals are referred to as the “electrical window” where a defibrillation shock is likely to be immediately effective, and where the provision of effective CPR is less crucially necessary for long term survival. Survival rates are poor because Emergency Medical Services personnel (activated via 911 Dispatch) usually take more than 10 min to arrive on scene and apply a defibrillator. Bystanders who access a nearby AED can save the victim's life by following simple instructions, which are typically audible and provided by the AED, and by applying a defibrillating shock from the AED to the victim. The AEDs are effective and lifesaving, even with minimal or no training, if applied quickly after the victim collapses, with survival rates increasing from 10-15% if an AED is not used, to 35-50% if an AED is connected to the victim and used effectively within approximately 5-10 minutes after onset of the cardiac arrest. Accordingly, when an AED is used, the survival rate triples. Alternatively, without any help to the victim within about 10 minutes after onset of cardiac arrest, there is virtually no chance of survival.

Unfortunately, AEDs are seldom deployed and used because bystanders cannot locate or retrieve the AED quickly enough. Therefore, even if bystanders know that an AED is required, locating and retrieving the AED is challenging. This is important since every minute of delay results in a 10% reduction in survival rates. For example, in Toronto, there are at least 20,000 AEDs but the problem is that in the heat of the moment bystanders forget to even search for one, or cannot find them since they are not clearly marked, which makes it difficult to retrieve an AED and bring it back to the scene of the cardiac emergency. Seeking out an AED may also prevent or delay effective CPR. To exacerbate this problem, if a bystander calls 911, the 911 dispatch usually has no idea where the nearest AED is. The AEDs are seldom registered, and even then the 911 dispatch may not know the precise location. The 911 dispatch alerts the EMS, who normally arrive in 10 minutes and then have to spend time looking for the victim. However, most of the time, the EMS arrives too late to save the victim.

Taking these factors into account, it's estimated that AEDs are used in only 5-10% of cardiac arrests in a public place in North America (and only in 11% of witnessed arrests), even though the AEDs may be available close by in the building (or nearby, even if outdoors) and in full public view. As a result, survival rates are about 17% for out-of-hospital witnessed cardiac arrests in a public place, in contrast to the 35-50% “save rate” if an AED is used.

### SUMMARY OF VARIOUS EMBODIMENTS

In one broad aspect, in accordance with the teachings herein there is provided an emergency assistance device for facilitating the delivery of emergency equipment to an emergency site, wherein the device is physically associated with the emergency equipment and the device comprises: a communication unit for sending and receiving electronic messages; at least one alert device for providing an alert to at least one passerby in visual or audible range of the at least one alert device; a GPS sensor; at least one motion sensor; memory comprising mapping software; and a processor that is operatively coupled to the communication unit, the at least one alert device, the memory, the at least one motion sensor and the GPS sensor, wherein the processor is configured to: control the at least one alert device to generate an alert when the communication unit receives a remote activation electronic message; generate and provide direction instructions to the at least one passerby using the GPS sensor and the mapping software when the processor detects, via the at least one motion sensor, that the device is picked up or moved by the at least one passerby; and provide usage instructions for using the emergency equipment at the emergency site when the processor determines that the device has arrived at the emergency site.

In at least one embodiment, the emergency equipment is an Automated External Defibrillator (AED), the emergency site is a victim location where a victim has suffered cardiac arrest and the usage instructions comprise instructions on applying the AED and optionally performing CPR on the victim.

In at least one embodiment, the processor is configured to generate at least one status update on its location while being transported to the victim location and use the communication unit to send the at least one status update to a remote device or server.

In at least one embodiment, the processor is configured to generate at least one status update on treatment of the victim and use the communication unit to send the at least one status update to a remote device or server.

In at least one embodiment, the device comprises a speaker and/or light source and the processor is configured to generate at least one control signal to control the speaker to generate a sound signal and/or to control the light source to generate a light signal.

In at least one embodiment, upon receiving the remote activation control message, the processor is configured to generate an electronic message that is transmitted by the communication unit to a device used by a registered lay rescuer, an emergency dispatcher, and/or an EMS person with instructions for performing one or more actions.

In another aspect, in accordance with the teachings herein, there is provided a computer implemented method for facilitating the delivery of emergency equipment to an emergency site wherein the device is physically associated with the emergency equipment and the method comprises: receiving via a communication unit at the device a remote activation electronic message; generating at least one of an audible and a visual alert to at least one passerby in the visual and/or audible range of the device using at least one alert device; generating and providing direction instructions to the at least one passerby, using a processor, at least one motion sensor, a GPS sensor and mapping software of the device, when the processor detects that the device is picked up or being moved by the at least one passerby from data obtained by the at least one motion sensor; and providing usage instructions for using the emergency equipment at the emergency site when the processor determines that the device has arrived at the emergency site.

In at least one embodiment, the emergency equipment is an Automated External Defibrillator (AED), the emergency site is a victim location where a victim has suffered cardiac arrest and the method comprises providing usage instructions for applying the AED and optionally performing CPR on the victim.

In at least one embodiment, the method comprises generating at least one status update on a location of the emergency assistance device using the processor and sending the at least one status update using the communication unit while the emergency assistance device is being transported to the victim location to a remote device or server.

In at least one embodiment, the method comprises generating at least one status update on treatment of the victim using the processor and sending the at least one status update using the communication unit to a remote device or server.

In at least one embodiment, upon receiving the remote activation control message, the method comprises generating an electronic message using the processor and transmitting the electronic message via the communication unit to a device used by a registered lay rescuer, a dispatcher and/or an EMS person with instructions for performing one or more actions.

In another aspect, in accordance with the teachings herein, there is provided a computer readable medium, comprising a plurality of instructions which, when executed on a processor, cause the processor to implement a method for facilitating the delivery of emergency equipment to an emergency site wherein the device is physically associated with the emergency equipment, wherein the method is defined in accordance with the teachings herein.

In another aspect, in accordance with the teachings herein, there is provided an emergency system for facilitating the delivery of emergency equipment to an emergency site, wherein the system comprises: a plurality of emergency assist devices that are associated with a plurality of emergency equipment devices; an emergency server that is in electronic communication with the plurality of emergency assist devices, the emergency server comprising: a communication unit for sending and receiving electronic messages to and from the plurality of emergency assist devices; a data store that comprises an emergency assist device database including data on the location of the emergency assist devices; and a processor that is operatively coupled to the communication unit and the datastore, wherein the processor is configured to: receive a location of an emergency site; locate the emergency assist devices that are within a predefined distance of the location of the emergency site; and send a remote activation electronic message to the located emergency assist devices.

In another aspect, there may be a multiplicity of methods to remotely activate the emergency assistance device, including but not limited to an automated signal that is generated when a wearable biologic sensor attached to the victim detects that the victim is undergoing or has experienced cardiac arrest or other serious heart rhythm or cardiac disorder, a manual or automatic activation from another individual who directly observes the victim undergoing or recently experiencing cardiac arrest or other serious heart rhythm or cardiac disorder, or the individual may receive and/or observe the automated signal that was triggered by the data recorded by the biological sensor where the automated signal is transmitted to a remote monitoring location at an institution or an emergency server.

In another aspect, in accordance with the teachings herein, there is provided a server for facilitating the delivery of emergency equipment to an emergency site, the server comprising: a communication unit for sending and receiving electronic messages to and from a plurality of emergency assist devices that are associated with a plurality of emergency equipment devices; a data store that comprises an emergency assist device database including data on the location of the emergency assist devices; and a processor that is operatively coupled to the communication unit and the datastore, wherein the processor is configured to: receive a location of an emergency site; locate the emergency assist devices that are within a predefined distance of the location of the emergency site; and send an remote activation electronic message to the located emergency assist devices.

In at least one embodiment, the processor is configured to access an EMS database to locate contact data for one or more EMS personnel and send an electronic message to a communication device of one or more EMS personnel to indicate the location of the emergency site.

In at least one embodiment, the processor is configured to access a registered user database to locate contact data for one or more registered lay rescuers and send an electronic message to a communication device of the one or more registered lay rescuers to indicate the location of the emergency site.

In at least one embodiment, the server is configured to receive a phone call, convert the phone call to text and parse the text to determine the location of the emergency site.

In at least one embodiment, the server is configured to receive an emergency electronic message, and parse text in the emergency electronic message to determine the location of the emergency site.

In at least one embodiment, the server is a remote emergency server or an institutional server.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a block diagram of an example embodiment of a system for emergency communication and a remotely activated emergency assist device in accordance with the teachings herein.

FIG. 2 shows a schematic diagram of an example embodiment of certain components of an emergency assist device that can be used in the system of FIG. 1 in accordance with the teachings herein.

FIG. 3 shows a flowchart of an example embodiment of a method for remote operation of a network of AEDs in accordance with the teachings herein.

FIG. 4 shows a flowchart of an example embodiment of a method for operation of an emergency assist device in accordance with the teachings herein.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, these terms can have a mechanical or electrical connotation such as indicating that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. the range of 1 to 5 includes all numbers from 1 to 5 such as, but not limited to, 1, 1.5, 2, 2.75, 3, 3.90, 4, and 5, for example). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

At least a portion of the example embodiments of the apparatuses or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including at least one of volatile and non-volatile memory). These devices may also have at least one input device (e.g., a touchscreen, and the like) and at least one output device (e.g., a display screen, a printer, a wireless radio, speakers or other sound generating device(s), light sources, and the like) depending on the nature of the device.

It should also be noted that there may be some elements that are used to implement at least part of the embodiments described herein that may be implemented via software that is written in a high-level procedural language such as object-oriented programming. The program code may be written in C, C++ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media (e.g., a computer readable medium such as, but not limited to, ROM, magnetic disk, optical disc) or a device that is readable by a programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Furthermore, it should be noted that reference to the figures is only made to provide an example of how various example hardware and software methods operate in accordance with the teachings herein and in no way should be considered as limiting the scope of the claimed subject matter.

Through their own research, the inventors have found that the impediments to finding, deploying, and using an AED include, in part, reluctance or lack of experience in an emergency by the bystanders, but most importantly, because the witnesses to the cardiac arrest need to begin CPR and call for help, and therefore they themselves have no opportunity or are unable to locate and retrieve the closest AED, which may be nearby, but not within line of sight. Furthermore, the witnesses to the cardiac arrest do not know how to alert individuals in the vicinity who may have training and skill in defibrillator use, and/or individuals who may know the location of the closest AED. If the AED is, for example, several hundred meters from the side of the victim, substantial time intervals may elapse until the AED can be brought to the victim's side. Every second counts, and in an emergency, where the immediate witnesses are busy doing CPR and calling 911, the task of finding, retrieving, and returning with an AED is stressful, time-consuming, and challenging. For the purposes of this description, the term “vicinity” is defined as a distance from an AED location from which the AED may be carried or delivered to the side of the victim and effectively deployed within a time period of less than 10 minutes.

In accordance with the teachings herein, example embodiments of a device, system and method are provided herein which facilitate the quicker delivery of the AED to the victim rather than a rescuer having to go locate the AED. This is implemented in such a way which allows for the most rapid delivery of the AED to the side of a cardiac arrest victim and involves the remote activation of the AEDs that are physically closest to the victim location and rapid effective deployment of the AED to the side of the victim. This may involve a witness accessing a software application on their mobile device which locates the closest AEDs and sends a remote activation electronic message to these located AEDs which in turn broadcast to passerby that there is a medical emergency and the AED needs to be delivered to the victim location within a certain period of time, such as 3 minutes, for example. Alternatively, the witness may use their mobile device to contact a central emergency server, in an organization, or emergency dispatch (e.g. via a public emergency system) which locates and remotely activates the closest AEDs.

In another aspect, in accordance with the teachings herein, example embodiments are provided herein which not only allow the AED to be delivered to the victim location but also alert one or more lay rescuers, that are trained in AED use and are also closest to the victim location, of the emergency situation (i.e. cardiac arrest), so that the lay rescuer may also arrive at the victim location in a very close time interval compared to the arrival of the AED at the victim location. The near simultaneous arrival of a lay rescuer who is trained in AED use, along with the arrival of the AED, within the “electrical window”, may substantially improve outcomes for Out-of-Hospital Cardiac Arrests (OHCAs). In the electrical window, provision of a defibrillation shock is likely to be immediately effective, making the provision of effective CPR is less crucially necessary for long term survival. The electrical window is about 3-5 minutes after the onset of the cardiac arrest and the collapse of the victim.

In another aspect, in accordance with the teachings herein, there is provided at least one example embodiment herein, which not only alerts the closest emergency medical services (EMS) personnel, such as paramedics, that there a cardiac arrest has occurred but also provides the exact victim location and the locations of the closest AEDs. This is important since EMS personnel are given the address that corresponds to the victim location but if the address is a building, or other structure, the EMS still have to physically locate the victim within the structure which can take time and reduces the chances that the AED or CPR is effectively provided to the victim in a timely fashion in order for the victim to survive. Furthermore, it remains unclear even to EMS dispatchers, how to inform the EMS personnel how far they may be away from the victim in a certain geographical area, specifically with respect to a building and which specific location within the building the victim may be located. Advantageously, if the AED carried by the EMS personnel also included an emergency assistance device, in accordance with the teachings herein, then they may be provided with verbal messages for how far they are away from a victim and which directions they should follow to reach the victim in the fastest amount of time.

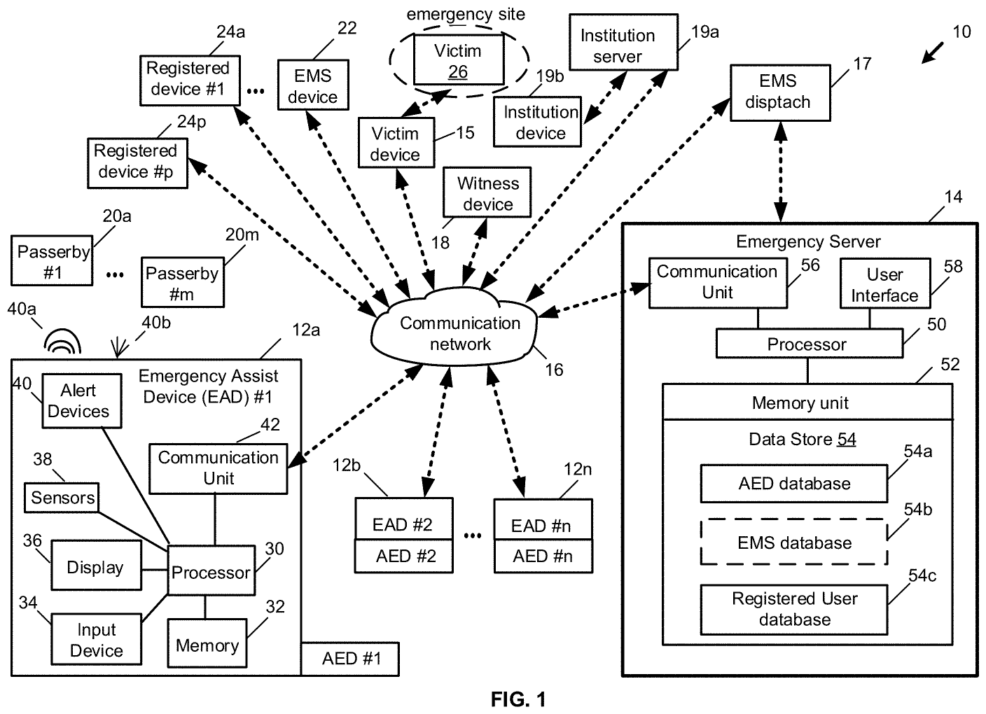

Referring now to FIG. 1, shown therein is a block diagram of an example embodiment of a system 10 for emergency communication and remotely activated emergency assist devices in accordance with the teachings herein. In this example embodiment, the system 10 comprises a plurality of Emergency Assist Devices or Emergency Assistance Devices (EADs) 12a to 12n, and an emergency server 14. The EADs 12a to 12n and the emergency server 14 communicate with one another through a communication network 16, which is generally a wireless network for cellular and/or Internet communication, for example. The EADs 12a to 12n are physically associated with AEDs.

The EADs 12a to 12n are devices that may be attached to a commercially existing AED or is integrated into the AED during manufacture of the AED. The EADs 12a to 12n can be used to broadcast (audibly and/or visually) to passersby when an OHCA occurs, with GPS-based written or audible location instructions to take the corresponding (i.e. attached) AED to the side of the victim. This will minimize communication delays inherent in the process of delivering an AED to the victim, potentially improving the survivability of an OHCA.

Taking EAD 12a as an example, the EAD 12a will generally be a small electronic device that may be the size of a cell phone with an enclosure that houses several components comprising a processor 30, a memory 32, an input device 34 (which may be optional), a display 36 (which may be optional), sensors 38, alert devices 40 and a communication unit 42. The EAD 12a may include further components needed for operation as is known by those skilled in the art such as a power unit (not shown) which can be any suitable power source that provides power to the various components of the EAD 12a such as a power adaptor or a rechargeable battery pack. It should be noted that in other embodiments, the EAD 12a may include other components. In at least some embodiments, the EAD 12a may also have redundant rechargeable battery packs to power the EAD 12a and optionally may receive power from the associated AED (i.e. the AED #1 to which the EAD 12a is operationally coupled) during operation during the rare occasions of power failures. For example, in at least some embodiments, the EAD 12a may employ a low power, polarized magnetic type DC connection that which will allow for easy pick up and transport of the EAD 12a during use.

The processor 30 may be a standard processor that controls the operation of the EAD 12a and becomes a specific processing device when executing certain programs that allow the EAS 12a to interact with passersby and the emergency server 14. The processor 30 may be implemented using a microcontroller or any other suitable processor with sufficient processing power for implementing the functionality of the EAD 12a.

The memory 32 generally includes RAM and ROM and is used to store an operating system and programs as is commonly known by those skilled in the art. For instance, the operating system provides various basic operational processes for the EAD 12a. The programs that are stored in the memory allow the EAD 12a to interact with various stakeholders as will be described in further detail below.

The input device 34 may be at least one of a touchscreen, a touchpad, a keyboard, a trackball, a microphone and the like depending on the particular implementation of the EAD 12a. For example, the input device 34 may provide a graphical user interface that allows a passerby or a technician to interact with the EAD 12a. In addition, or in an alternative embodiment, the user of the EAD 12a may provide verbal commands which are sensed by the microphone, translated to text, parsed to determine the content of the verbal command and then processed to take action based on the verbal command.

The display 36 can be any suitable display that provides visual information and may be implemented using an LCD-based display, using available technology, which may or may not have touchscreen ability. The display 36 may be used by the processor 30 to provide messages to a passerby who picks up the AED 12a to bring it to the victim site or to provide status messages regarding the operational status of the AED #1 to which the EAD 12a is operatively coupled with. For example, an operational status message may be displayed such as whether certain components of the AED #1 need to be serviced and/or whether the battery of AED #1 needs to be recharged or replaced. For example, an electronic warning message may be generated by the processor 30 and transmitted via the communication unit 42 and the communication network 16 to the emergency server 14 at pre-set times (e.g. at monthly, quarterly or yearly intervals like every 1- or 2-years) that batteries used by the AED are due for replacement).

The communication unit 42 may be a standard wireless network communication device such as a wireless transceiver that communicates utilizing CDMA, GSM, GPRS, Bluetooth (such as Blue Tooth Low Energy (BLE)) or ZigBee according to standards such as IEEE 802.11a, 802.11b, 802.11g, or 802.11n. The communication unit 42 can provide the processor 30 with a way of communicating with other devices or computers including the emergency server 14 and possibly directly with user devices such as the witness device 18, registered user devices 24a to 24p and/or the EMS device 22. The witness device 18, registered user devices 24a to 24p and/or the EMS device 22 may be referred to as communication devices (e.g. smartphones or cell phones with data transmission capabilities).

The sensors 38 are used by the processor 30 in order to determine the surroundings of the EAD 12a which will help the EAD 12a to direct a passerby to the victim location and also to detect movement of the EAD 12a. For example, the sensors 38 can include a GPS sensor in order to determine the location of the EAD 12a and at least one motion sensor such as an accelerometer and/or gyroscope to detect when the EAD 12a has been moved/picked up by a person. The processor 30 may operate a map software module which then uses the current location of the EAD 12a and the victim location to determine the directions needed to bring the passerby with the AED #1 to the victim location using the shortest and/or fastest possible route. The processor 30 then provides these directions to the passerby via the display 36 and/or a speaker (e.g. provided by alert devices 40).

For example, in some embodiments, the processor 30 may execute an existing mapping software application with this capability and the processor 30 may display a map on the display 36 with directions to the victim location. In some cases, the map may be very detailed and indicate the vicinity surrounding the AED including all hallways, corridors, street level maps, within building directions, and the like, in an area that is approximately a 10 minute rapid walk or jog from the defibrillator “home location”. In addition, in some embodiments, the processor 30 may display a Graphical User Interface (GUI) showing the location of the various actors or stakeholders which are persons who are within the “circle of care” (i.e. have activated the emergency system or have received communication from the emergency system, e.g. registered users, EMS personnel, victim, witness and bystanders) to help address the cardiac arrest situation. In some embodiments, the cell phone number of the victim may be known to the actors and may be tracked remotely (this may be useful in cases where the witness has to leave the scene and a person wants to know where the victim has been taken or in case the victim was moved and the victim's phone moves along with the victim).

In at least one embodiment, the EAD 12a may also include a USB port for installation of mapping software to perform the various mapping and directional functions described herein. In other instances the USB port may be used for performing software upgrades on the EAD 12a or performing diagnostics.

It should also be noted that since the EAD 12a includes a GPS sensor, the EAD 12a can be instantaneously tracked and tied with a specific AED manufacturer or model. This tracking also allows EMS dispatch 17, through the emergency server 14, to track the movement of the AED as it is being brought to the victim location. The emergency server 14 can then provide periodic electronic updates to the witness device 18 about the expected time arrival (ETA) of the AED to the victim location.

The sensors 38 may also include operational sensors such as a current sensor and/or a voltage sensor that the processor 30 may interact with to determine the operational status of the AED #1 to which the EAD 12a is operationally coupled. For example, the current and voltage sensors may be used by the processor 30 to determine if the batteries of the AED #1 are in sufficiently good condition to allow the AED #1 to provide electrical defibrillation signals as required in an emergency. If the batteries need maintenance or replacement, the processor 30 may send an electronic message to the emergency server 14 using the communication unit 42 and the communication network 16 to indicate that the AED #1 needs maintenance.

The sensors 38 may include other sensors for obtaining data that is useful in attending to the victim. For example, the sensors 38 may include a camera and a microphone for audio and visual data to be recorded and sent to the emergency server 14 or to an EMS device 22 so that personnel with the appropriate medical training can view the data and provide additional instructions to the person who is administering the AED and/or CPR to the victim. The sensors 38 may also include an accelerometer and motion sensors that can be used to determine movement of the EAD 12a and therefore movement of the associated AED #1.

In at least one embodiment, the sensors 38 may also include pressure sensors that may be attached to the patient (i.e. victim) to track blood flow and thus the biological (i.e. physiological) state of the victim. In at least one embodiment, the sensors 38 may alternatively or additionally include motion sensors that can be affixed to the victim and track chest movements (i.e. respiration) and body movement of the victim, which can help the EMS personnel and/or EMS dispatch 17 assess the health state of the victim.

In at least one embodiment, the sensors 38 may also, or alternatively, include a proximity sensor that provides data on the proximity of the EAD 12a to its associated AED #1. This may be useful in situations in which the EAD 12a is separated from its associated AED #1 and therefore maintenance may be required to attach the EAD 12a to its associated AED #1.

In at least one embodiment, the sensors 38 may also, or alternatively, include a temperature sensor and/or an ambient gas sensor, such as a carbon monoxide sensor. This may be useful in situations where the EAD 12a and its associated AED #1, or another associated piece of medical or safety equipment, is being taken to the scene of a fire.

The alert devices 40 can be used to alert individuals (i.e. passersby) within the vicinity of the AED #1 such as those people that are in visual line of sight or earshot of AED #1 that a cardiac arrest has occurred, and that the AED #1 needs to be transported to the side of the victim (i.e. the victim location). Accordingly, the alert devices 40 include various electronic components that can be used to generate sounds 40a such as a speaker and/or to generate lights 40b such as an LED or other light source. The generated sound 40a and lights 40b are alert signals to indicate that the EAD 12a is in “emergency mode”. The light alert 20b may be provided by flashing lights, or strobe lights. In both cases, the processor 30 may generate control signals that are then amplified and applied to the corresponding hardware components. Accordingly, after alerting one of the passersby, the EAD 12a can provide audio instructions to give clear indications to the passerby for where the site of cardiac arrest is located and how to take the AED there (via the GPS tracking and mapping application) when the passerby has picked up the AED and shown an interest to taking the AED to the victim site. For example, the EAD 12a may provide a verbal or visual instruction to the passerby to press a certain button on the housing of the EAD 12a or on its display so that the passerby can confirm that they will bring the AED along with the EAD 12a to the victim location. Alternatively, this may happen automatically as the EAD 12a senses that it is being used based on data obtained by its GPS and/or internal motion sensor such as an accelerometer and/or gyroscope. Further instructions can be provided by the EAD 12a when the passerby arrives at the victim location such as, for example, “Undo the zipper, remove the AED from its bag, remove the plastic covering from the electrode pads, and press the green ON button on the AED to turn it on and follow additional instructions that may be accompany the AED”.

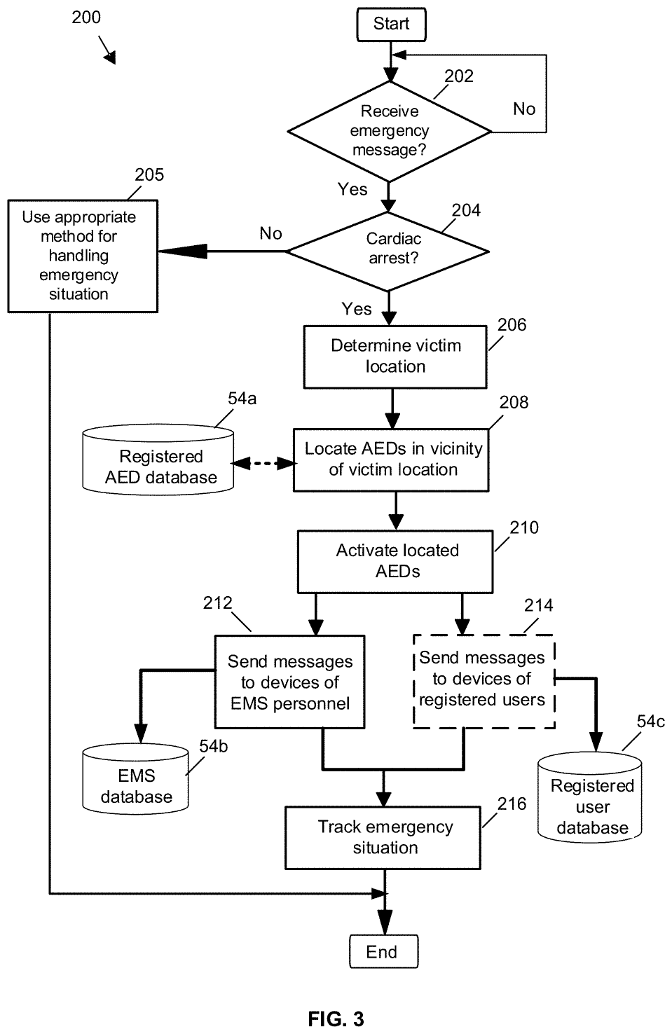

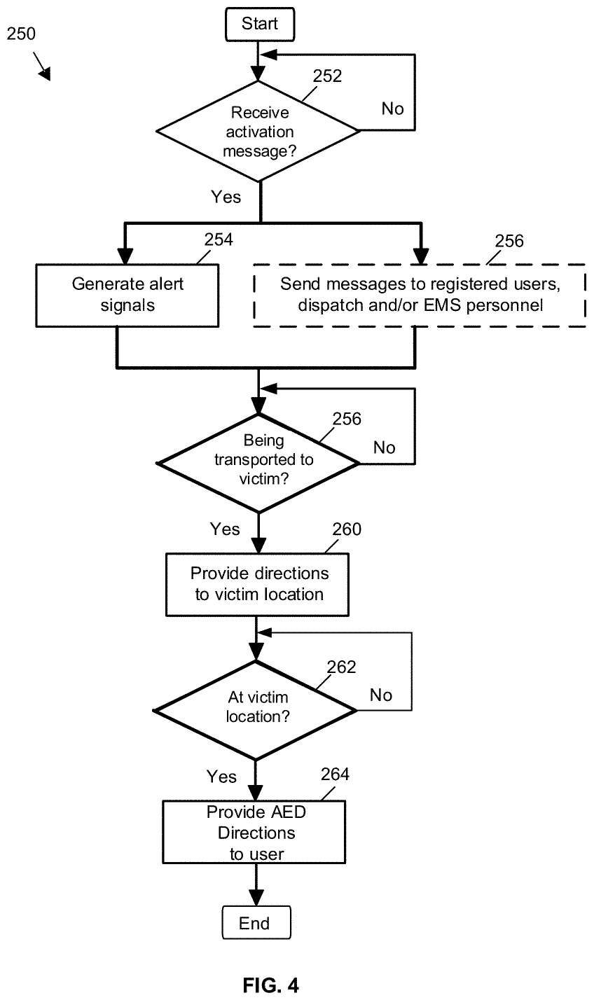

For example, in at least one embodiment, data for a human or synthesized voice command may be stored in the memory 32 and be sent by the processor 30 through a digital to analog converter (not shown) to the speaker (not shown) in order to generate the audio alert which indicates that the AED #1 needs to be delivered to the side of the victim. For example, if the EAD 12a is in an airport an example of the audio alert is: “Emergency! Emergency! There is a cardiac arrest nearby! You can save a life! Please pick me up and carry me to gate 50!”. Depending on the implementation of the system 10 and the EADs, the audio alert may indicate that EMS personnel have been notified and any local registered individuals have been notified as explained further with respect to methods 200 and 250 in FIGS. 3 and 4, respectively.

In at least one embodiment, the processor 30 may also be configured for generating an electronic EMS message and sending the electronic EMS message via the communication unit 42 and the communication network 16 to mobile devices associated with EMS personnel that are on route to the victim location, an example of which is shown as EMS device 22 in FIG. 1. The electronic EMS message may include data about the victim location, the status of the AED being transported to the victim location and/or the condition of use of the AED.

In at least one embodiment, the processor 30 may also be configured for generating an electronic “lay alert” message and sending the electronic lay alert message via the communication unit 42 and the communication network 16 to mobile or stationary (i.e. land line) devices 24a to 24p, for example, that are associated with previously specified trained lay rescuer(s) that have registered with the emergency system 10. These registered trained lay rescuers may be living or working in the vicinity of the particular EAD that has been remotely activated and they are prepared to respond in cases of emergency. For example, in some cases, the AEDs, along with their associated EAD, that are local to the victim location and have been remotely activated are owned by an institution or company where the cardiac arrest occurred. The institution may be a building, an arena, an organization like a business, college, university or other school, or an airport, etc. and the registered lay rescuers may be security guards or employees of the institution. The electronic lay alert message generated by the processor 30 can indicate that cardiac arrest has occurred and that the registered lay rescuers are to pick up a particular AED and deliver it to the side of the victim and use the AED as they were previously trained to do so.

Alternatively, in some embodiments, the registered lay rescuers may be given specific tasks to perform, as indicated in the electronic alert message that is sent to their mobile devices when a cardiac arrest has occurred. For example, the electronic alert message may include a first set of instructions to be sent to a device of one of the registered lay rescuers who is tasked with getting the AED and bringing it to the victim location, another electronic alert message may include a second set of instructions and be sent to a device of a registered lay rescuer who is tasked with going to the victim location and performing CPR and yet another electronic alert message may include a third set of instructions and be sent to a device of a registered lay rescuer who is tasked with going to the entrance of the facility to wait for the arrival of EMS personnel and bring the EMS personnel to the victim location.

In at least one alternative embodiment, the system 100 can also include an institution server 19a, (connected to the communication network 16) and a person, such as a witness and/or employee at the institution, can use their device 18, which might be their electronic mobile device such as a smart phone, or a device 19b provided by the institution such as an emergency button that is connected to the institution's emergency system, to send an emergency electronic message to the institution server 19a to alert members of the institution when a cardiac event has occurred. The institution server 19a may also communicate via the communication network 16 to other stakeholders and/or the emergency server 14. In this case, the emergency electronic message includes location data for the location of the witness device 18 or the institution device 19b. In some cases, the witness may tell an employee, such as an attendant or security guard for example, who works at the institution about the cardiac event and the employee then sends the emergency electronic message, using their mobile device or the institution device, to the institution server. The institution server 19a runs a software program, which may be similar to the activation software program used by the server 14, that upon receipt of the emergency electronic message, is capable of determining the closest AEDs that are in the vicinity of where the emergency electronic message originated and send a remote activation signal to the closest AEDs and their associated EADs, which may then operate as described herein. In some cases, the institution server 19a may also send one or more electronic notification messages to one or more personnel at the institution who can assist with providing medical assistance to the victim who has experienced the cardiac event. In such embodiments, the response time can be quicker than having to contact the emergency services server 14 (which may be via activation of the local Emergency response system “911”), which is described in further detail below. In some cases, the institution server 19 may also contact 911 and/or EMS dispatch 17.

In at least one embodiment, a lock or other means to secure the AED may be used to prevent tampering or theft. For example, the lock may be used to securely lock a container housing for the AED and then, later upon receiving an unlock signal via remote activation, the lock may be unlocked so that the container is AED can be accessed. The unlock signal which may be remotely sent by the emergency server 14, institution server 19a, or other designated individuals. Upon unlocking of the container, the passerby (i.e. rescuer) can gain access to the AED to take it to the victim location.

In at least one embodiment, in circumstances where the EAD 12a is not activated (for example as part of routine device surveillance), the processor of the EAD 12a may generate and transmit electronic messages to the emergency server 14 or the institutional server 19a to verify that the EAD 12a and its attached AED #1 are physically intact (i.e. still in a locked or closed container and not tampered with). The electronic message may also include the current location of the EAD 12a. These electronic messages may be generated and transmitted at pre-determined time intervals.

In at least one embodiment, at the time of first installation of the EAD to a corresponding AED, the processor 30 may generate and transmit an electronic registration message to the emergency server 14 and/or institutional server 19a. The electronic registration message can include the geolocation of the EAD (and thus the location of the AED) as well as the operational status of the AED. The emergency server 14 and/or institutional server 19a may comprise software for storing registration information for the EAD as well as its location and the operational status of the associated AED in a database such as AED database 54a.

In another aspect, in accordance with the teachings herein, there is provided a central emergency server 14 that can be accessed by a witness to the cardiac arrest to request emergency medical attention. Access to the emergency server 14 may be done in a few ways using the system 10. For example, the witness may use their mobile device (i.e. witness device 18), to contact emergency dispatch (i.e. EMS dispatch 17). The EMS dispatch 17 may then access the emergency server 14 to assist with the remote activation of the EAD 12a. The EMS dispatch 17 may be a person that the witness may call using their device 18 which may be a mobile device or a landline. Alternatively, in some cases the EMS dispatch 17 may be automated and the witness may send an electronic message to the emergency server 14 that indicates that a cardiac arrest has occurred.

Upon determination that the medical emergency is for a victim who has suffered from cardiac arrest, the emergency server 14 determines the victim location from the electronic message sent by the witness device 18 or in some cases a call made by the witness. The emergency server 14 may use an activation software program to access a database of registered AEDs 54a, and remotely activate the registered AEDs that are closest to the victim location, such as the AEDs that are within a certain radius, such as within 500 meters of the victim site, for example. The emergency server 14 can also perform one or more other tasks as will be evident from the following description and the description of FIGS. 3 and 4. For example, the emergency server 14 may execute a tracking software program that operates in conjunction with GPS technology to track the location of an activated AED that is being taken to a victim location.

The emergency server 14 comprises a processor 50, a memory 52, a data store 54, a communication unit 56 and a user interface 58. In other embodiments, the emergency server 14 may include other components. The processor 50 and the communication unit 56 may be implemented in a similar manner as the processor 30 and the communication unit 42 of the EAD 12a. However, the processor 50 and the communication unit 56 may use processing and communication components that allow for greater processing and transmitting capacity as the emergency server 14 may communicate with many different devices that are at many different distances from the physical location of the server 14.

The processor 50 is operatively coupled to the memory unit 52 that will store the operating system and programs for allowing the emergency server 14 to perform various functions including remote activation of AEDs, tracking of AEDs, registration of AEDs, and updating various databases that are contained in the data store 54. For example, the data store 54 may include an AED database 54a, an EMS database 54b (which is optional) and a registered user database 54c.

The AED database 54a includes the location as well at least one of a description and/or visual image of all of the AEDs that have been registered with the emergency server 14 for the area that is covered by the emergency system 10. The AED database 54a may also include information on the operational status of the registered AEDs, the owner of the AEDs, AED product information (e.g. model number, manufacturer and the like) and/or personnel charged with its upkeep or maintenance.

The EMS database 54b may include data on the EMS workers that can be dispatched by the emergency system 10, such as the contact details (e.g. phone numbers and/or email addresses) for the EMS workers. It should be noted that in some cases the information that will be included in the EMS database 54b may be proprietary in which case it is not stored on the data store 54 and the emergency server 14 may then communicate with a separate EMS system in order for emergency dispatch to contact the EMS personnel that may be associated with the system 10 in order to dispatch them.

The registered user database 54c contains contact, identify and location information for the registered lay rescuers who are registered with the emergency system 10. For example, the registered lay users may be people who are trained to use AEDs and/or provide CPR and want to help if there are any cardiac arrests near their work location or their residence. In other cases, where AEDs are owned by certain institutions, e.g. corporations or organizations, the registered lay rescuers may be designated people that are employees, such as security guards or emergency staff, who are tasked with aiding in a medical emergency situation.

The user interface 58 may be a GUI that allows an operator, such as a dispatch person or other emergency personnel, to operate the emergency server 14 to carry out the various functions of the emergency system 10 described herein. For example, the GUI can have various windows for providing data to the operator and also text boxes or other input elements (i.e. virtual buttons) for allowing the operator to provide control inputs to the emergency server 14. For example, the GUI can allow the operator to see the location of an incoming call (and therefore the victim location), determine the AEDs that are nearest to the victim location, remotely activate the AEDs that are within a certain distance of the victim location, and also contact EMS to go to the victim location. The GUI may also allow the operator (i.e. emergency dispatcher) to view the progress of a rescue and track the arrival of the AED and the EMS personnel to the victim location. In an alternative embodiment, the emergency server 14 may be programmed to perform the aforementioned functions in an automated fashion.

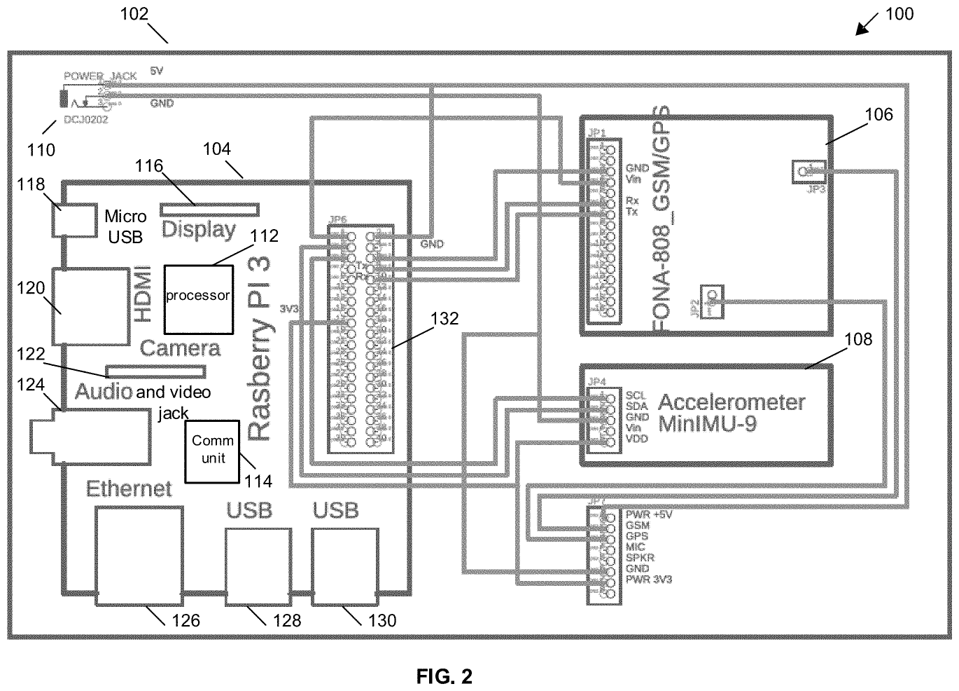

Referring now to FIG. 2, shown therein is a schematic diagram of an example embodiment of certain components for an emergency assist device 100 that can be used in the system of FIG. 1 in accordance with the teachings herein. The EAD 100 includes a first circuit board 102 for receiving and connecting various circuit components such as, but not limited to, a processing circuit board 104, a GPS 106, an accelerometer 108 and a power jack 110 for connection to a power source. In this example embodiment, the processing circuit board (e.g. a motherboard) 104 includes a processor 112, a communication unit 114, a display port 116, a micro USB port 118, an HDMI port 120, a camera port 122, an audio and video jack 124, an Ethernet port 126, a first USB port 128, a second USB port 130, and a General purpose 10 port 132. The light source that is used to provide the emergency alert light signal may be connected to the GPIO port 132 or another suitable port. It should be noted that other implementations may be used for the internal electronics of an EAD.

Referring now to FIG. 3, shown therein is a flowchart of an example embodiment of a method 200 for remote operation of a network of AEDs using an emergency system in accordance with the teachings herein. The emergency system may be implemented as emergency system 10 and the emergency system may be automated or there may be an operator, such as an emergency dispatcher, for receiving emergency calls or electronic messages, remotely activating certain AEDs and deploying EMS to the victim location. For ease of illustration, the description of FIG. 3 will assume that the emergency system 10 is automated and controlled by one or more programs running on an emergency server, such as emergency sever 14. It should be noted that the method of FIG. 3 may be used with the institution server 19a may have similar components as the emergency server 14 and may operate in a similar manner as the emergency server 14.

At act 202, the emergency server is waiting to receive an emergency call or an emergency electronic message (e.g. a text message, an SMS, an email, and the like). During this time, the AEDs associated with the emergency system are in a low power standby mode. The emergency call or emergency electronic message can be sent by a witness device 18 (via a witness such as a bystander), a device used by an institutional worker, by the victim's device 15 through a trigger caused by data sensed by a biologic sensor of the victim where the data is indicative of cardiac arrest or other cardiac emergency and the occurrence of the trigger causes the automatic generation and transmission of the emergency electronic message, or by an individual who has access to the data recorded by the biologic sensor (for example when the victim is being monitored via telemetry. In some cases, the emergency message is received by 911 dispatch or EMS dispatch who then triggers/interacts with the emergency server.

In embodiments where the victim's device 15 generates the emergency electronic message, the victim's device 15 may be connected directly or wirelessly to a wearable device and a software application along with sensors, such as electrodes, is used that is capable of detecting when the victim suffers a cardiac arrest and automatically sends the emergency electronic message to any or all of the emergency server 14, the institutional server 19a, emergency dispatch, or to AEDs which the software application has determined are closest to the victim's location.

When an emergency message is received at act 202, it is then determined whether the emergency situation involves a cardiac arrest. If the emergency situation does not involve a cardiac arrest then the method 200 proceeds to act 205 where the appropriate method is used for handling this particular non-cardiac emergency situation. For example, a speech to text program and a text parser may be used to convert incoming audio calls into text, or a text parser may be used for incoming electronic messages having text, where in both cases the text parser analyzes the text to determine whether the emergency situation involves a cardiac arrest by looking for certain keywords or phrases such as “collapse”, “not moving or breathing”, “cardiac”, “cardiac arrest”, and/or “heart attack”. Alternatively, an operator may review the call or electronic messages. In either case, when it is determined that the received emergency message relates to a cardiac arrest, the method 200 proceeds to act 206.

At act 206, the emergency server 14 will determine the victim location. The emergency server may automatically display the geographic location coordinates of the victim of the cardiac arrest (from information provided by the witness who is calling 911, or with a location system based on the witness device 18 (e.g. cell signal, WiFi signal, Bluetooth signal) or land line of the witness contacting the emergency dispatch if there is an operator that is interacting with the emergency server 14.

At act 208, the emergency server 14 will locate the nearest AEDs to the victim location. This may involve locating the AEDs that are within a particular radius, such as within 200, 300 or 500 meters of the victim location. This emergency server 14 may access the registered AED database 54a in order to locate the AEDs that are within the defined radius of the victim location and which are also in good working condition.

At act 210, the emergency server 14 may then send an electronic activation message to the EADs that are associated with the located AEDs via the communication unit 56 and the communication network 16. At this point the located EADs and AEDs are activated and may operate in accordance with one or more aspects of the teachings herein. For example, a method of operation for the activated EADs and associated AEDs is provided by method 250 in FIG. 250.

At act 212, the emergency server 14 may then send electronic messages to the devices of the EMS personnel, such as EMS device 22, to go to the victim location. The contact information for the EMS personnel may be obtained via the EMS database 54c.

At act 214, the emergency server 14 may then send electronic messages to the devices 24a to 24p of registered lay rescuers that are in close proximity to the victim location and the activated EADs and activated AEDs. For example, the registered lay rescuers may be people that are trained to use AEDs and provide CPR who live or work in the vicinity of the victim location. Alternatively, the registered lay rescuers may be people that are employed by an organization that owns the AEDs and who will provide certain functions in the case of a cardiac emergency as described previously.

It should be noted that acts 212 and 214 may be performed in a different order or they may be performed in parallel. In an alternative embodiment, act 214 may be optional.

At act 216, the emergency server 14 may track the progress of the emergency situation. This may include tracking the location of the AED that is being brought to the victim location through a GPS tracking system as described previously. This tracking may also include tracking the location of any dispatched EMS personnel and any dispatched registered lay rescuers.

Referring now to FIG. 4, shown therein is a flowchart of an example embodiment of a method 250 for operation of an Emergency Assist Device (EAD) in accordance with the teachings herein. The operation of only one EAD will be described (i.e. EAD 12a) in relation to FIG. 4 for ease of illustration. However, it should be noted that multiple EADs that are within a predefined vicinity of the victim location can be remotely activated at the same time and operate in a similar manner.

At act 252, the EAD 12a is waiting to receive an electronic activation signal or an electronic activation message from one of several devices. For example, the EAD 12a may receive the electronic activation message from the emergency server 14 based on the location of the AEDs and the victim's GPS coordinates. Alternatively, the EAD 12a may receive the electronic activation message from an institutional server 19a, an institutional employee or an EAD of another AED. For example, in at least one embodiment, an activated EAD may send electronic messages to activate other designated EADs that are located in the vicinity (i.e. within 500 to 1000 m) but where these EADs are not activated by the emergency server 14 or the institutional server 19a to provide redundancy. This may mean for example, that all EADs in one building, on one floor of a building, in one area of a building, etc. may be activated by one of the EADs. In another embodiment, if the emergency server 14 or the institutional server 19a is not functioning properly, but any bystander picks up any AED physically, a first EAD attached to the AED may be configured to automatically activate the nearby EADs and instruct these nearby EADs to provide instructions to follow the first EAD to the victim site. In the absence of activation, the EAD 12a is in low power standby mode in order to conserve power. In addition, in the absence of activation, the EAD 12a may periodically broadcast its status (e.g. active, disabled, error in functioning, low battery, etc.) which may be separate from scheduled status communications. Once the EAD 12a receives the electronic activation message, which includes the victim location, the EAD 12a awakens and the method 250 proceeds to act 254.

The electronic activation message may be provided by the emergency server 14. Alternatively, in some embodiments, the witness device 18 may have a direct AED activation software program, which the witness can use to directly communicate with the EAD associated with the closest AED rather than having to go through the emergency server 14 or call dispatch. In this case the direction activation software being used by the witness device 18 may be implemented in a similar manner as the locating and activation software employed by the emergency server 14 or institutional server 19a. The closest EAD 12a may then be activated by the witness pressing a button on a GUI that is displayed on their device 18 and associated with the direct activation software program.

At act 254, the EAD 12a generates alert signals to passersby to indicate that there is an emergency situation nearby and that the AED which is associated with the EAD 12a must be taken to the victim location. For example, the EAD 12a may first alert individuals (i.e. passersby 20a to 20m) within the vicinity of the associated AED using loud beacon or siren noises and then indicate that an OHCA has occurred and that the AED needs to be transported to victim. The audio announcement may also include the type, nature, and location of the emergency and that the AED must be transported to the emergency site (i.e. victim location) as quickly as possible. The audio message may be broadcast in English and/or another language which may be a predefined language based on where the AED is located. At this point a remote unlock electronic message may be sent to the activated AEDs to temporally unlock the enclosures that contain the AEDs to allow a passerby access to the AED in order to take it to the victim location.

At act 256, the EAD 12a may optionally send electronic messages to other stakeholders associated with the emergency system 10 such as EMS personnel, registered lay rescuers and/or the emergency server 14 to indicate the exact location of the victim as well (as the location of the AED when it is being brought to the victim location).

At act 258, the EAD 12a determines that it has been “picked up” and is being taken to the victim location. This may be determined through various sensors that are employed by the EAD 12a to determine its geographical location, orientation and movement. For example, the EAD 12a typically has a GPS sensor, and an accelerometer that can be used to track its movement. At this point, the EAD 12a may broadcast a new message such as: “A cardiac arrest victim has been identified and is located at XXXX” where XXXX is the specific location of the cardiac arrest victim. At this point, the EAD 12a may also send an electronic message to the emergency server 14/EMS dispatcher 17 to indicate that it has been picked up and is being carried to the side of the victim. The emergency server 14/EMS dispatcher 17 can then send an electronic message to alert bystanders on site (and optionally EMS personnel) that the AED is “on its way”. In an alternative embodiment, the EAD 12a may also communicate directly through a wireless connection and the communication network 16 with the devices of the bystanders on scene (e.g. who have identified themselves at the time of the original EMS call) to indicate to them that the AED is soon to arrive on scene (an ETA may be provided as well in some embodiments). When the EAD 12a determines that it has been picked up by a rescuer and is being transported to the victim location, the method 250 proceeds to act 258.

In at least some embodiments, if the EAD 12a determines that the individual picking up the AED is not a previously designated trained lay rescuer, the EAD 12a may send a separate electronic message via the communication unit 42 and the communication network 16 to the registered device(s) used by one or more registered trained lay rescuers to indicate that it has been picked up by somebody other than a designated trained rescuer. This then allows the registered trained lay rescuer to proceed directly to the side of the victim instead of the location where the AED is normally stored.

At act 260, the EAD 12a provides location directions to the rescuer to direct the rescuer to the victim location. The directions may be audible instructions and/or visual instructions that are provided on the display of the EAD 12a in the formal of a map and/or text instructions. The directions are preferably the shortest and/or fastest route to the cardiac arrest location, which may be implemented using currently known mapping technology. Since the EAD 12a employs both GPS hardware and other communication hardware including cellular radios, this allows for indoor and outdoor use in navigating to the victim's site and communicating with the other stakeholders including the EMS dispatch 17 and/or the emergency server 14. Furthermore, in at least some embodiments, the owner of an AED may designate one or more designated persons/places (locations, cell numbers) to be called in an emergency, which may be done by the EAD 12a. The designated persons may include front line workers, the owner of the facility which houses the AED, security personnel, health/rescue personnel and communications personnel (e.g., front desk, etc.).

During transit, the EAD 12a may optionally periodically send electronic messages to other stakeholders associated with the emergency system 10 such as the EMS personnel, registered lay rescuers and/or the emergency server 14 to indicate the location of the AED as it is being brought to the victim location. The provision of real-time communication with EMS dispatch and/or the emergency server 14 as well as instructions on AED deployment on route will improve the chances of saving the victim. The transmission of these electronic messages may be sent periodically to keep all stakeholders up-to-date on the transport of the AED to the victim location. The EAD 12a may also send data in these electronic update messages regarding the status and condition of use of the AED and may also provide audible updates through the speaker as the EAD 12a determines it is close to the victim location via its GPS sensor. In at least some embodiments, the EMS dispatch 17 and/or the emergency server 14 may also alert the witness that the AED is being brought to the victim location and provide an approximate ETA.

At act 262, the EAD 12a continually checks to determine when it arrives at the victim location. This may be done through a mapping program and the GPS sensor that are employed by the EAD 12a as described previously to determine when the EAD 12a is within a few meters of the victim location. Once the EAD 12a determines that it is at the victim location the method 250 proceeds to act 264.

In an alternate embodiment, the witnesses to the cardiac arrest, via their device 18, can remotely cause the EAD to send a message or signal to the AED, and activate the AED, causing it, for example, to “turn on” and the covering of the pre-gelled pads to spontaneously come free from the pads themselves by motorized or other means, to prepare them more expeditiously for application to the chest of the victim. This may be done as the AED is being delivered to the victim location and this may be done under instructions from the emergency server 14 to the witness device 18.

At act 264, the EAD 12a is at the victim location and begins to broadcast audio instructions or display visual instructions including text on how the AED can be effectively deployed to the victim and optionally how to perform effective CPR on the victim. For example, the instructions can tell the user of the AED how to deploy the AED by opening its case or other packaging, turning the AED on, removing the pre-gelled defibrillator pads from their housing, removing the backing from the pre-gelled pads, removing the victim's clothing to allow the pre-gelled pads to be placed directly on the victim's chest, and activating the appropriate sequence of button presses on the AED to make a rhythm analysis/diagnosis and deliver electrical shocks if necessary. The instructions may also include instructing bystanders on the best methods for performing CPR, which may be done by the speaker or display, for example. The EAD 12a will thus begin to “talk through” the bystanders even before the pads are connected to the victim. These steps are not always obvious or clearly marked on conventional AEDs and only after a conventional AED is deployed on the patient, the conventional AED then starts to monitor cardiac rhythm and gives subsequent instructions.

In some embodiments, depending on the circumstances the EAD 12a may be in cellular communication with the EMS dispatch 17 which will allow for two two-way communication between the EMS dispatch 17 and the user of the EAD 12a so that EMS dispatch can also provide verbal commands and feedback to facilitate the effective use of the AED and CPR.

These instructions may be continued until the cardiac arrest has been dealt with. The EAD 12a may also broadcast update electronic messages during this time to the various stakeholders that are not at the victim location to update them on the actions that are currently being performed on the victim such as CPR or delivery of defibrillation electrical signals. For example, in at least one embodiment, the EAD 12a may also include one or more sensors for biological signal acquisition such as, but not limited to, an oxygen saturation monitor, which can be put on the victims finger. Such a device allows for both pulse detection, heart rate measurement, and oxygen saturation measurement. The EAD 12a may have a port attached to a wire attached to the oxygen saturation sensor which is placed on the victims finger tip, and data corresponding to the pulse, heart rate and/or oxygen saturation can be measured and recorded by the EAD 12a and transmitted to any and all relevant actors before and/or when the EMS is at the victim site.

Accordingly, the various embodiments of the devices, systems and methods described herein employ wireless functionality of the EADs as well as the communication network 16, the server 14, the institution server 19a (depending on the embodiment) and the various electronic devices to allow for multi-way communication (e.g. two-way, three-way or more) between the EADs, the server 14, the EMS dispatch 17 who remotely activated the device, bystanders at the scene of the emergency, a designated lay rescuer, and/or the witness to the cardiac arrest. In another aspect, the EADs may make outgoing calls as well as incoming calls or electronic transmission and reception of electronic messages.

It should be noted that in other embodiments, an autonomous machine, such as a drone, a robot or an autonomous vehicle, may be remotely alerted along with the EAD and used to bring the EAD along with the AED to the victim's location. In some embodiments, the EAD may include navigation software that directly controls the autonomous machine, or is connected to a manual or automated remote controller of the autonomous machine, to control the movement of the autonomous machine to direct it to the victim's location.

Therefore, to summarize, the teachings herein provide fora way to minimize the communication and transport delays inherent in the process of finding, identifying, retrieving, and transporting an AED to a cardiac victim's location. Using GPS, and locator technology, including cellular signals, and potentially WiFi and Bluetooth, for contacting EMS personnel and/or other registered lay rescuers, the other time delays that are encountered in rescue situations can be occurring concurrently resulting in no additional delays (i.e. no serial accumulation of the delays) as multiple personnel can be electronically messaged at the same time (which also ensures redundancy in case some potential rescuers are unavailable). By reducing the time that it takes for the AED to arrive to the side of the victim, the survivability can thus be substantially improved.

The various embodiments of the device, system and methods described herein may be particularly useful for dealing with cardiac arrests that occur in enclosed spaces, such as in hockey arenas, shopping malls, exercise venues, sports clubs, company offices and entertainment complexes. The several minutes required to locate and retrieve an AED will be significantly reduced using the teachings herein. It is estimated that the normal 6-8 minutes required to deploy an AED may be cut down to 3-4 minutes using the teachings herein which may result in a 30-40% increase in the likelihood of survival, using the generally accepted “every minute delay results in 10% lower survival”.

It should also be noted that in other embodiments, the system 10 may be used for other types of emergencies, in addition to or instead of cardiac emergencies, which will require other types of medical equipment including, but not limited to, stretchers, oxygen tanks, and first aid equipment, for example, as well as other types of emergency equipment such as, but not limited to, ladders, fire extinguishers, chemical retardants and heavy equipment to extricate a person from a confined space, for example. In such embodiments, the EADs 12a to 12n can be attached to these different emergency equipment items and the system 10 may generally operate in a similar manner as has been described for AEDs. Also, in such embodiments, the EADs may also be programmed to make other types of announcements including emergency announcements, public announcements and/or evacuation orders.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. An emergency assistance device for facilitating the delivery of emergency equipment to an emergency site, wherein the device is physically associated with the emergency equipment and the device comprises:
a communication unit for sending and receiving electronic messages;
at least one alert device for providing an alert to at least one passerby in visual or audible range of the at least one alert device;
a GPS sensor;
at least one motion sensor;
memory comprising mapping software; and
a processor that is operatively coupled to the communication unit, the at least one alert device, the memory, the at least one motion sensor and the GPS sensor, wherein the processor is configured to:
control the at least one alert device to generate an alert when the communication unit receives a remote activation electronic message;
generate and provide direction instructions to the at least one passerby using the GPS sensor and the mapping software when the processor detects, via the at least one motion sensor, that the emergency assistance device is picked up or being moved by the at least one passerby; and
provide usage instructions for using the emergency equipment at the emergency site when the processor determines that the device has arrived at the emergency site.

2. The device of claim 1, wherein the emergency equipment is an Automated External Defibrillator (AED), the emergency site is a victim location where a victim has suffered cardiac arrest and the usage instructions comprise instructions on applying the AED and optionally performing CPR on the victim.

3. The device of claim 2, wherein the processor is configured to generate at least one status update on its location while being transported to the victim location and use the communication unit to send the at least one status update to a remote device or server.

4. The device of claim 2, wherein the processor is configured to generate at least one status update on treatment of the victim and use the communication unit to send the at least one status update to a remote device or server.

5. The device of claim 1, wherein the device comprises a speaker and/or light source and the processor is configured to generate at least one control signal to control the speaker to generate a sound signal and/or to control the light source to generate a light signal.

6. The device of claim 1, wherein upon receiving the remote activation control message, the processor is configured to generate an electronic message that is transmitted by the communication unit to a device used by a registered lay rescuer, an emergency dispatcher, and/or an EMS person with instructions for performing one or more actions.

7. A computer implemented method for facilitating the delivery of emergency equipment to an emergency site wherein the device is physically associated with the emergency equipment and the method comprises:
receiving via a communication unit at the device a remote activation electronic message;
generating at least one of an audible and a visual alert to at least one passerby in the visual and/or audible range of the device using at least one alert device;
generating and providing direction instructions to the at least one passerby, using a processor, a GPS sensor, at least one motion sensor and mapping software when the processor detects that the device is picked up or being moved by the at least one passerby from data obtained by the at least one motion sensor; and
providing usage instructions for using the emergency equipment at the emergency site when the processor determines that the device has arrived at the emergency site.

8. The method of claim 7, wherein the emergency equipment is an Automated External Defibrillator (AED), the emergency site is a victim location where a victim has suffered cardiac arrest and the method comprises providing usage instructions for applying the AED and optionally performing CPR on the victim.

9. The method of claim 8, wherein the method comprises generating at least one status update on a location of the emergency assistance device using the processor and sending the at least one status update using the communication unit while the emergency assistance device is being transported to the victim location to a remote device or server.

10. The method of claim 8, wherein the method comprises generating at least one status update on treatment of the victim using the processor and sending the at least one status update using the communication unit to a remote device or server.

11. The method of claim 7, wherein upon receiving the remote activation control message, the method comprises generating an electronic message using the processor and transmitting the electronic message via the communication unit to a device used by a registered lay rescuer, a dispatcher and/or an EMS person with instructions for performing one or more actions.

12. A server for facilitating the delivery of emergency equipment to an emergency site, the server comprising:
a communication unit for sending and receiving electronic messages to and from a plurality of emergency assist devices that are associated with a plurality of emergency equipment devices;
a data store that comprises an emergency assist device database including data on the location of the emergency assist devices; and
a processor that is operatively coupled to the communication unit and the datastore, wherein the processor is configured to:
receive a location of an emergency site;
locate the emergency assist devices that are within a predefined distance of the location of the emergency site; and
send a remote activation electronic message to the located emergency assist devices.

13. The server of claim 12, wherein the processor is configured to access an EMS database to locate contact data for one or more EMS personnel and send an electronic message to a communication device of one or more EMS personnel to indicate the location of the emergency site.

14. The server of claim 12, wherein the processor is configured to access a registered user database to locate contact data for one or more registered lay rescuers and send an electronic message to a communication device of the one or more registered lay rescuers to indicate the location of the emergency site.

15. The server of claim 12, wherein the server is configured to receive a phone call, convert the phone call to text and parse the text to determine the location of the emergency site.

16. The server of claim 12, wherein the server is configured to receive an emergency electronic message, and parse text in the emergency electronic message to determine the location of the emergency site.

17. The server of claim 12, wherein the server is a remote emergency server or an institutional server.
